(19) United States
(12) Patent Application Publication
(10) Pub. No.: US 2014/0011839 A1
(43) Pub. Date: Jan. 9, 2014

(54) COMPOSITIONS AND METHODS FOR TREATING AND INHIBITING VIRAL INFECTIONS

(71) Applicant: Justice E. OBI, St. Albans, NY (US)

(72) Inventor: Justice E. OBI, St. Albans, NY (US)

(21) Appl. No.: 13/932,445

(22) Filed: Jul. 1, 2013

Related U.S. Application Data
(63) Continuation-in-part of application No. 13/687,273, filed on Nov. 28, 2012.
Application No. 13/687,273 is a continuation-in-part of application No. 13/540,749, filed on Jul. 3, 2012.
(60) Provisional application No. 61/825,569, filed on May 21, 2013.

(51) Int. Cl. A61K 31/4706

(52) U.S. Cl. CPC A61K 31/4706
USPC 514/313; 546/163

(57) ABSTRACT

Compositions and methods for the treatment, as well as the inhibition and prevention, of an infection of the papillomavirus and the epithelial lesions, namely, the warts of the skin and mucosal surfaces, associated therewith, in a mammalian host, as well as methods of inhibiting the replication of a papillomavirus in an infected cell, are provided. The compositions comprise a therapeutically effective amount of an active ingredient comprising at least one compound selected from the group consisting of chloroquine, hydroxychloroquine, amodiaquine, or in each case, a pharmaceutically acceptable salt thereof. The methods comprise topically administering a therapeutically and/or antivirally effective amount of such a compound to a mammalian host, such as a human being, in need of such treatment, although alternatively other routes of administration may be used, including but not limited to transdermal, transmucosal, respiratory, and by injection. The compositions optionally also comprise one or more pharmaceutically acceptable non-active ingredients.

### REFERENCE TO RELATED APPLICATIONS

This application is a continuation-in-part of prior co-pending application Ser. No. 13/687,273, filed Nov. 28, 2012, which was a continuation -in-part of prior co-pending application Ser. No. 13/540,749, filed Jul. 3, 2012, This application also claims the benefit under 35 U.S.C. §119(e) of prior U.S. Provisional Patent Application Serial No. 61/825,569, filed May 21, 2013.

### TECHNICAL FIELD

This invention relates, in general, to compositions and methods for treating and inhibiting papillomavirus infections and removing warts of papillomavirus genesis in mammalian hosts, and in particular, to the treatment and prevention of genital warts and other warts and lesions of the skin and mucosal surfaces that are associated with infections of the papillomavirus in mammals. More specifically, this invention relates to compositions and methods for treating and inhibiting replication of the human papillomarivus, thereby eliminating the cause of such warts and lesions.

### BACKGROUND OF THE INVENTION

The human papillomavirus is a virus of the papillomavirus family that infects the epidermis of human beings. Such infections may result in epithelial lesions or growths, particularly warts, also known as condylomas or papillomas. Many different human papillomavirus types have been identified, which are usually transmitted through skin-to-skin contact (or mucous membrane contact), including sexual contact. Human papillomavirus types 6 and 11 are most often associated with unsightly and embarrassing, but usually non-cancerous, warts on the male genitalia, in and around the vagina, in and around the anus, in and around the throat, including the larynx, or other skin and/or mucous membrane areas. Other human papillomavirus types include, but are not limited to, types 16 and 18, which are also transmitted through skin-to-skin contact (or mucous membrane contact), including sexual contact, and are associated with unsightly and often embarrassing anogenital warts. The human papillomavirus is also known generally to play a role in and may in fact lead to, several types of cancers, including cervical, vaginal, vulvar, penile, anal, rectal and/or oropharyngeal cancers, although types 16 and 18 are associated with a higher risk of cancer than types 6 and 11 and other types. Other human papillomavirus types include type 1, which is associated with unsightly but usually non-cancerous warts that appear on the feet. The present invention may be used to treat and inhibit infections caused by all of the aforementioned human papillomavirus types, but the invention is not limited to the treatment and inhibition of infections caused solely by the human papillomavirus types mentioned above,

Infections caused by the human papillomavirus, and warts associated with that virus, have often been treated and/or removed using one of the following compositions and/or methods: cryotherapy, which involves freezing the abnormal cells with liquid nitrogen; conization, or a cone biopsy, which removes the abnormal areas surgically; or Loop Electrosurgical Excision Procedure (LEEP), where the abnormal cells are removed with an electrical current. For warts in less sensitive areas, acids may be applied to assist in terminating the wart; canadid antigen may be injected to stimulate the immune system to fight off the wart; a topical medication called imiquimod may be applied; or no treatment at all may be used, which implies simply waiting to see if the cells can heal on their own.

However, many of these compositions and/or methods include burning or freezing away the wart (using chemicals or an electrical current), which can often be a painful process, and in any event can leave unsightly scarring. Others utilize a medication that modifies the immune response to the virus in order to treat the wart, but these often require more time to fight the virus and terminate the wart, which does not always happen. Additionally, while these methods may be used to target and treat existing warts, they do not necessarily treat the underlying virus which may result in a recurrence of the warts, as the warts are a manifestation of the viral infection.

It is therefore one of the primary objects of the present invention to provide compositions and methods for treating infections caused by the human papillornavirus and for removing warts associated with that virus, particularly, but not limited to, those associated with types 1, 6, 11, 16 and 18.

Another object of the present invention is to provide compositions and methods for treating infections caused by the human papillomavirus and for removing warts associated with that virus in which the compositions are safe enough to be used topically on highly sensitive regions of the skin, such as may be found in the genital areas.

Still another object of the present invention is to provide compositions and methods for treating infections caused by the human papillomavirus and for removing warts associated with that virus which can be administered to humans through different routes of administration and in a variety of forms.

Yet another object of the present invention is to provide compositions and methods for treating infections caused by the human papillomavirus and for removing warts associated with that virus which can also be used to cover scars left after conventional surgical removal of such warts, in order to prevent re-growth of such warts.

Still one other object of the present invention is to provide compositions and methods for treating infections caused by the human papillomavirus and for removing warts associated with that virus which provide quick relief and termination of the wart, as well as sustained relief to prevent additional warts from forming, by inhibiting replication of virus.

### SUMMARY OF THE INVENTION

The present invention provides compositions and methods for treating an infection associated with the human papillomavirus: such an infection may be manifested in a human as epithelial lesions, including warts, and particularly skin warts, lesions and anogenital warts. The compositions of the invention for treating and removing such warts comprise at least one compound selected from the group consisting of chloroquine, hydroxychloroquine and amodiaquine. More particularly, the compositions comprise either chloroquine alone or hydroxychloroquine alone or amodiaquine alone, or pharmaceutically acceptable salts thereof, as the active ingredient, or combinations of any two, or all three, of those compounds, or pharmaceutically acceptable salts thereof, as the active ingredients. Optionally, the compositions may also comprise suitable pharmaceutically acceptable carriers, excipients and/or adjuvants, and/or other non-active ingredients, such as ethyl alcohol, lidocaine, epinephrine and/or diphenhydramine. It is believed that the compositions of the invention act by inhibiting replication of human papillorravirus which causes such warts.

The methods of the invention comprise administering a pharmaceutical composition comprising at least one compound selected from the group consisting of chloroquine, hydroxychloroquine, amodiaquine and pharmaceutically acceptable salts thereof, to a human being, using one or more routes of administration that are well known in the art. At present, the following are the preferred routes of administration: topical (epicutaneous), in forms such as a gel, cream, spray, soap or other bathing apparatus; transdermal, in the form of a patch; transmucosal (also known as pharmaceutical pessary delivery) through the vagina or rectum, in the form, for example, of an ovule or suppository, respectively; nasal, such as delivery by inhalation of aerosol droplets produced, for example, with the aid of a nebulizer; and infusion under the skin in the form of an injection; Other routes for administering pharmaceutical compositions according to the invention are possible

Thus, one aspect of the present invention generally concerns methods for the treatment of a human or mammalian subject suffering from an existing papillomavirus infection by inhibiting further replication of the virus. In one embodiment of this aspect, the methods comprise administering a therapeutically effective amount of chloroquine or a pharmaceutically acceptable salt thereof to the subject. In another embodiment the methods comprise administering a therapeutically effective amount of hydroxychloroquine or a pharmaceutically acceptable salt thereof to the subject. In yet another embodiment the methods comprise administering a therapeutically effective amount of amodiaquine or a pharmaceutically acceptable salt thereof to the subject. In still another embodiment of this aspect of the invention, the methods comprise administering a therapeutically effective amount of chloroquine or a pharmaceutically acceptable salt thereof together with a therapeutically effective amount of hydroxychloroquine or a pharmaceutically acceptable salt thereof to the subject. In yet another embodiment, the methods comprise administering a therapeutically effective amount of chloroquine or a pharmaceutically acceptable salt thereof together with a therapeutically effective amount of amodiaquine or a pharmaceutically acceptable salt thereof to the subject. In still another embodiment, the methods comprise administering a therapeutically effective amount of hydroxychloroquine or a pharmaceutically acceptable salt thereof together with a therapeutically effective amount of amodiaquine or a pharmaceutically acceptable salt thereof to the subject. In yet another embodiment, the methods comprise administering a therapeutically effective amount of chloroquine or a pharmaceutically acceptable salt thereof together with a therapeutically effective amount of hydroxychloroquine or a pharmaceutically acceptable salt thereof and a therapeutically effective amount of amodiaquine or a pharmaceutically acceptable salt thereof to the subject.

Another aspect of the invention generally concerns methods for the prevention or inhibition of the development or recurrence of the development in a human or mammalian subject of an infection of the human papillomavirus by inhibiting replication of the virus. In one embodiment of this aspect, the methods comprise administering an antivirally effective amount of chloroquine or a pharmaceutically acceptable salt thereof to the subject. In another embodiment the methods comprise administering an antivirally effective amount of hydroxychloroquine or a pharmaceutically acceptable salt thereof to the subject. In still anther embodiment the methods comprise administering an antivirally effective amount of arnodiaquine or a pharmaceutically acceptable salt thereof to the subject. In still another embodiment of this aspect of the invention, the methods comprise administering an antivirally effective amount of chloroquine or a pharmaceutically acceptable salt thereof together with an antivirally effective amount of hydroxychloroquine or a pharmaceutically acceptable salt thereof to the subject. In yet another embodiment, the methods comprise administering an antivirally effective amount of chloroquine or a pharmaceutically acceptable salt thereof together with an anitvirally effective amount of amodiaquine or a pharmaceutically acceptable salt thereof to the subject. In still another embodiment, the methods comprise administering an antivirally effective amount of hydroxychloroquine or a pharmaceutically acceptable salt thereof together with an antivirally effective amount of amodiaquine or a pharmaceutically acceptable salt thereof to the subject. In yet another embodiment, the methods comprise administering an antivirally effective amount of chloroquine or a pharmaceutically acceptable salt thereof together with an antivirally effective amount of hydroxychloroquine or a pharmaceutically acceptable salt thereof and an anitvirally effective amount of amodiaquine or a pharmaceutically acceptable salt thereof to the subject.

Another aspect of the invention generally concerns pharmaceutical compositions for the treatment of a papillomavirus infection in a human or mammalian host by inhibiting replication of the virus. In one embodiment of this aspect, the compositions comprise a therapeutically effective amount of chloroquine or a pharmaceutically acceptable salt thereof. In another embodiment the compositions comprise a therapeutically effective amount of hydroxychloroquine or a pharmaceutically acceptable salt thereof. In still another embodiment the compositions comprise a therapeutically effective amount of amodiaquine or a pharmaceutically acceptable salt thereof. In still another embodiment of this aspect of the invention, the compositions comprise a therapeutically effective amount of chloroquine or a pharmaceutically acceptable salt thereof together with a therapeutically effective amount of hydroxychloroquine or a pharmaceutically acceptable salt thereof. In yet another embodiment, the compositions comprise a therapeutically effective amount of chloroquine or a pharmaceutically acceptable salt thereof together with a therapeutically effective amount of amodiaquine or a pharmaceutically acceptable salt thereof. In still another embodiment, the compositions comprise a therapeutically effective amount of hydroxychloroquine or a pharmaceutically acceptable salt thereof together with a therapeutically effective amount of amodiaquine or a pharmaceutically acceptable salt thereof. In yet another embodiment, the compositions comprise a therapeutically effective amount of chloroquine or a pharmaceutically acceptable salt thereof together with a therapeutically effective amount of hydroxychloroquine or a pharmaceutically acceptable salt thereof and a therapeutically effective amount of amodiaquine or a pharmaceutically acceptable salt thereof.

Another aspect of the invention generally concerns methods for the treatment of a papillomavirus infection in a cell of human or mammalian origin, as well as methods of inhibiting the replication of a papillornavirus in a cell of human or mammalian origin infected with such a virus. In one embodiment of this aspect, the methods comprise exposing the cell to an antivirally effective amount of chloroquine or a pharmaceutically acceptable salt thereof. In another embodiment the methods comprise exposing the cell to an antivirally effective amount of hydroxychloroquine or a pharmaceutically acceptable salt thereof. In yet another embodiment the methods comprise exposing the cell to an antivirally effective amount of amodiaquine or a pharmaceutically acceptable salt thereof. In still another embodiment of this aspect of the invention, the methods comprise exposing the cell to an antivirally effective amount of chloroquine or a pharmaceutically acceptable salt thereof together with an antivirally effective amount of hydroxychloroquine or a pharmaceutically acceptable salt thereof. In yet another embodiment, the methods comprise exposing the cell to an antivirally effective amount of chloroquine or a pharmaceutically acceptable salt thereof together with an antivirally effective amount of amodiaquine or a pharmaceutically acceptable salt thereof. In still another embodiment, the methods comprise exposing the cell to an antivirally effective amount of hydroxychloroquine or a pharmaceutically acceptable salt thereof together with an antivirally effective amount of amodiaquine or a pharmaceutically acceptable salt thereof. In yet another embodiment, the methods comprise exposing the cell to an antivirally effective amount of chloroquine or a pharmaceutically acceptable salt thereof together with an antivirally effective amount of hydroxychloroquine or a pharmaceutically acceptable salt thereof and an antivirally effective amount of amodiaquine or a pharmaceutically acceptable salt thereof.

These and other aspects, features, objects and advantages of the present invention will become more apparent to those skilled in the art from the following detailed description of the presently most preferred embodiments thereof.

### DETAILED DESCRIPTION OF THE PREFERRED EMBODIMENT

The present invention resides in the discovery that the known compounds chloroquine, hydroxychloroquine and amodiaquine, or pharmaceutically acceptable salts thereof, all of which have been used previously as antimalarial agents and/or to treat disorders of the immune system, also have utility in treating infections of the human and other mammalian papillomaviruses, and in particular, in treating and in inhibiting replication of such viruses and in removing the warts associated with such infections, as well as in preventing the recurrence of such warts.

The molecular structures of chloroquine, hydroxychloroquine and amodiaquine are provided below, as Formula (I), Formula (II) and Formula (III), respectively.

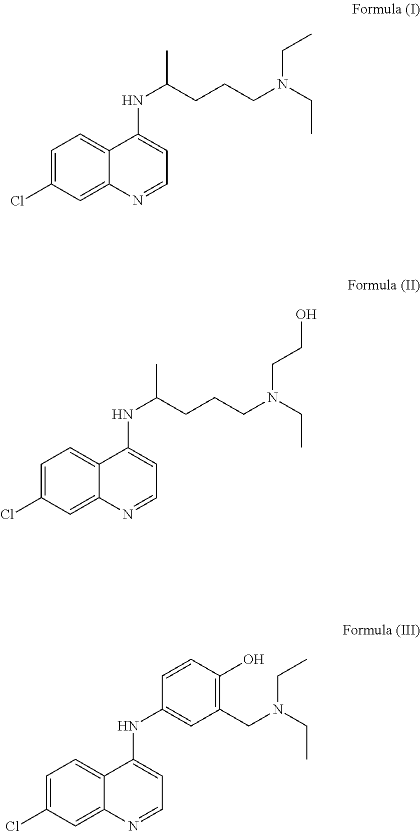 [SMILES: CCN(CC)CCCC(C)Nc1ccnc2cc(Cl)ccc12 ; CCN(CCO)CCCC(C)Nc1ccnc2cc(Cl)ccc12 ; CCN(CC)Cc1cc(Nc2ccnc3cc(Cl)ccc23)ccc1O]

As is known, and as can be seen in the structural formulas set out above, these three compounds are related to one another in that their structures have a heterodicyclic portion in common, namely, the 7-chloro derivative of the 4-aminoquinoline moiety. Chloroquine (Formula I) has been marketed under the brand name Resochin, while hydroxychloroquine (Formula II) has been marketed under the brand name Plaquenil, and amodiaquine (Formula III) has been marketed under the brand name Camoquin. Chloroquine is commercially available as the pharmaceutically acceptable salt chloroquine phosphate, and may be purchased in solid (tablet) form from a wide variety of sources such as Ohm Laboratories, Inc of North Brunswick, N.J., U.S.A., while a ready-made aqueous solution may be purchased from a wide variety of sources, including Sal Parenterals (P) Ltd., of Hyserabad, India, or Scott Edil Pharmacia Ltd., of Jhannajri, India. Hydroxychloroquine, in solid (tablet) form, is commercially available as the pharmaceutically acceptable salt hydroxychloroquine sulphate, and may be purchased from a wide variety of sources such as West-Ward Pharmaceutical Corporation of Eatontown, N.J., U.S.A. Amodiaquine, in solid (tablet) form, is commercially available as the pharmaceutically acceptable salt amodiaquine hydrochloride, and may be purchased from a wide variety of sources, including Parke, Davis & Company, a division of Pfizer Inc., headquartered in New York, N.Y., U.S.A.

In the preferred embodiments, the compositions of the invention comprise either chloroquine (Formula I) alone or hydroxychloroquine (Formula II) alone or amodiaquine (Formula Hi) alone as the active ingredient, or either chloroquine combined with hydroxychloroquine, or chloroquine combined with amodiaquine, or hydroxychloroquine combined with amodiaquine, or chloroquine combined with both hydroxychloroquine and amodiaquine, as the active ingredients, in general, as mentioned above and as set forth in further detail below, the methods of the invention comprise, in the preferred embodiments, administering the pharmaceutical compositions of the invention comprising the active ingredient or ingredients to a mammal such as a human being using one or more of the preferred routes of administration, which include direct topical (epicutaneous) administration, in forms such as gel, cream, lotion, solution, spray, soap or other bathing apparatus; transdermal administration, in the form of a patch; transmucosal administration (also known as pharmaceutical pessary delivery) through the vagina or rectum, in the form, for example, of an ovule or a suppository, respectively; respiratory administration, such as delivery via inhalation through the nostrils and nasal passages of aerosol droplets produced, for example, with the aid of a nebulizer; and infusion under the skin in the form of an intra-epidermal injection.

Although the compositions and methods of the invention will be illustratively described hereinafter with reference to topical, transdermal and infusional routes of administration, it should be understood that the invention is not limited to the specific cases described, but extends also to the use of other compatible routes for administering pharmaceutical compositions according to the invention, as will be evident to those skilled in the art, including but not limited to other topical and/or parenteral routes, such as buccal, conjunctival, endotracheal, intramuscular, intravascular, laryngeal, or ophthalmic, or even enteral (oral) routes, any one or more of which may ultimately be found to be more preferable. Suitable formulations for any route of administration that is ultimately selected are known and are described in well-known texts, including for example Remington, The Science and Practice of Pharmacy, 21^{st} edition, 2005, Mack Publishing Company, Easton, Pa., and therefore such formulations may easily be prepared by those of ordinary skill in the art.

It is well understood, however, that proper dosages of any medication may vary from one individual to another, depending on many factors such as the intensity of the affliction and the selected route of administration, as well as the weight, age and gender of the patient. Therefore, the effective dosages of the pharmaceutical compositions of the present invention should be determined by a specialist in this matter, such as a medical doctor or other health care provider, depending on these and other parameters. Nevertheless, for the sake of illustration only, exemplary formulations, preparation procedures and dosages, for topical, transdermal and infusional routes of administration, are provided below for the sake of guidance.

When the compositions of the invention comprising chloroquine as the only active ingredient are to be administered topically (epicutaneously), in the form of either a gel or a spray, the compositions preferably comprise chloroquine plus optional non-active ingredients. The non-active ingredients may comprise ethyl alcohol and peppermint spirit oil, and the gel form may additionally comprise lidocaine jelly or ointment, while the spray form may additionally comprise lidocaine as a 2% solution. All of the non-active ingredients are conventional and are available commercially from a wide variety of sources The non-active ingredients are exemplary only, but are included for the following purposes: the lidocaine functions as an analgesic, the ethyl alcohol functions as an antiseptic, while the peppermint spirit oil functions to provide a pleasing odor. A particularly preferred composition for the gel form may be prepared from the starting ingredients set forth below:

| Chloroquine phosphate | 4000 | mg (8 500-mg tablets) |
|---|---|---|
| Lidocaine jelly 2% or ointment 5% | 10 ml or 10 mg, respectively | |
| Ethyl alcohol (70% aqueous solution) | 15 | ml |
| Peppermint spirit oil | 2.5 | ml |
| Water | 2.5 | ml |

Preferably, this composition is prepared by crushing the chloroquine phosphate tablets in a mortar, and then adding each of the non-active ingredients, in the sequence listed above, sufficient to make a total of approximately 30 gm of the gel or spray. The gel form may then be transferred to a tube or other appropriate container, while the spray form may then be transferred into a conventional spray bottle.

The gel form of this medication is preferably administered to a patient as follows, The affected areas are first cleaned with one or more alcohol swabs, and optionally, each wart to be treated may then be filed lightly for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) by improving the contact between the wart and the medication. Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

When the compositions of the invention comprising chloroquine as the only active ingredient are to be administered transdermally, in the form of a patch, approximately 3.5 gm of the gel form (prepared as described above) may be transferred to the pad of a conventional 3.5 mini-patch. This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

When the compositions of the invention comprising chloroquine as the only active ingredient are to be administered in injectable form, the compositions preferably comprise an aqueous solution of chloroquine plus optional non-active ingredients. The non-active ingredients may comprise diphenhydramine, lidocaine solution and epinephrine. A particularly preferred composition is set forth below:

| | |
|---|---|
| Chloroquine phosphate solution | 20 ml |
| Lidocaine (2% solution) | 5 ml |
| Epinephrine Injection 1:1000 | 2.5 ml |
| Diphenhydramine 50 mg/ml | 2.5 ml |

Each milliliter of the chloroquine phosphate solution contains 64.5 mg B.P. (British Pharmacopia) of chloroquine phosphate, which is equivalent to 40 mg of chloroquine. All of the non-active ingredients are conventional and are available commercially from a wide variety of sources. The non-active ingredients are included for the following purposes: the lidocaine functions as a local anaesthetic, the diphenhydramine functions as an antihistimine to decrease any possible allergic reaction to the active ingredient, while the epinephrine functions to constrict small blood vessels. Preferably, this composition is prepared by mixing the active ingredient with the non-active ingredients (in any sequence), so as to yield a sufficient amount of the injectable form of the medication to fill a 30 ml vial.

The injectable form of this medication is preferably administered to a patient as follows. Tuberculin syringes, each having a capacity of 3 ml to 5 ml, are used to withdraw aliquots of 0.5 ml to 1 ml from the 30 ml vial. This amount is then injected (with appropriate sterilization) into or beneath the base of each wart or infected area, once a day for 5-7 days, until disappearance of the wart(s) or infection. Although administration by injection is the fastest-acting route of administration for the medication, it is the preferred route of administration only for warts or infections appearing in one or more body cavities or on the hard surfaces of the skin, such as the toes or other parts of the foot, the fingers or other parts of the hand, the elbows and the knees; it is not the preferred route of administration for warts appearing on the male genitalia. Further, although the preparation of an injectable form has been described above only with respect to compositions of the invention comprising chloroquine as the sole active ingredient, those of ordinary skill in the art will be capable of formulating injectable forms of the other compositions of the invention described below, including those comprising hydroxychloroquine alone or amodiaquine alone, and including those comprising more than one of the three active ingredients.

When the compositions of the invention comprising hydroxychloroquine as the only active ingredient are to be administered topically (epicutaneously), in the form of either a gel or a spray, the compositions preferably comprise hydroxychloroquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form may be prepared from the starting ingredients set forth below:

| Hydroxychloroquine sulphate | 2400 | mg (12 200-mg tablets) |
|---|---|---|
| Lidocaine jelly 2% or ointment 5% | 10 ml or 10 mg, respectively | |
| Ethyl alcohol (70% aqueous solution) | 15 | ml |
| Peppermint spirit oil | 2.5 | ml |
| Water | 2.5 | ml |

Preferably, this composition is prepared by crushing the hydroxychloroquine sulphate tablets in a mortar, and then adding each of the non-active ingredients, in the sequence listed above, sufficient to make a total of about 30 gm of the gel or spray. The gel form may then be transferred to a tube or other appropriate container, while the spray torn may then be transferred into a conventional spray bottle.

The gel form of this medication is preferably administered to a patient topically as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s). Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage for the spray form is preferably two puffs applied to the affected areas once or twice daily.

When the compositions of the invention comprising hydroxychloroquine as the only active ingredient are to be administered transdermally, in the form of a patch, approximately 3.5 gm of the gel form (prepared as described above) may be transferred to the pad of a conventional 3.5 mini-patch, This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

When the compositions of the invention comprising amodiaquine as the only active ingredient are to be administered topically (epicutaneously), in the form of either a gel or a spray, the compositions preferably comprise amodiaquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form is set forth below:

| Amodiaquine hydrochloride | 800 | mg (4 200-mg tablets) |
|---|---|---|
| Lidocaine jelly 2% or ointment 5% | 10 ml or 10 gm, respectively | |
| Ethyl alcohol (70% aqueous solution) | 15 | ml |
| Peppermint spirit oil | 2.5 | ml |
| Water | 2.5 | ml |

Preferably, this composition is prepared by crushing the amodiaquine hydrochloride tablets in a mortar, and then adding each of the non-active ingredients, in the sequence listed above, sufficient to make a total of approximately 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed lightly for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) by improving the contact between the wart and the medication. Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

When the compositions of the invention comprising amodiaquine as the only active ingredient are to be administered transderrnally, in the form of a patch, approximately 3.5 gm of the gel form (prepared as described above) may be transferred to the pad of a conventional 3.5 mini-patch. This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

When the compositions of the invention comprising chloroquine in combination with hydroxychloroquine as the active ingredients are to he administered topically (epicutaneously), in the form of either a gel or a spray, the compositions preferably comprise chloroquine and hydroxychloroquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form is set forth below:

| Chloroquine phosphate | 4000 | mg (8 500-mg tablets) |
|---|---|---|
| Hydroxychloroquine sulfate | 2400 | mg (12 200-mg tablets) |
| Lidocaine jelly 2% or ointment 5% | 10 ml or 10 gm, respectively | |
| Ethyl alcohol (70% aqueous solution) | 15 | ml |
| Peppermint spirit oil | 2.5 | ml |
| Water | 2.5 | ml |

Preferably, this composition is prepared by crushing the chloroquine phosphate tablets together with the hydroxychloroquine sulfate tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops), only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks. until disappearance of the vvart(s). The spray form of this medication is preferably administered to a patient in the same manner, although the dosage is preferably two puffs applied to the affected areas once or twice daily.

When the compositions of the invention comprising chloroquine in combination with hydroxychloroquine as the active ingredients are to be administered transdermally, in the form of a patch, approximately 3.5 gm of the gel form (prepared as described above) may be transferred to the pad of a conventional 3.5 mini-patch. This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

When the compositions of the invention comprising chloroquine in combination with amodiaquine as the active ingredients are to be administered topically (epicutaneousiy), in the form of either a gel or a spray, the compositions preferably comprise chloroquine and amodiaquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form may be prepared from the starting ingredients set forth below:

| Chloroquine phosphate | 4000 | mg (8 500-mg tablets) |
|---|---|---|
| Amodiaquine hydrochloride | 800 | mg (4 200-mg tablets) |
| Lidocaine jelly 2% or ointment 5% | 10 ml or 10 mg, respectively | |
| Ethyl alcohol (70% aqueous solution) | 15 | ml |
| Peppermint spirit oil | 2.5 | ml |
| Water | 2.5 | ml |

Preferably, this composition is prepared by crushing the chloroquine phosphate tablets together with the amodiaquine hydrochloride tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops), only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

When the compositions of the invention comprising chloroquine in combination with amodiaquine as the active ingredients are to be administered transidermally, in the form of a patch, approximately 3.5 gm of the gel form (prepared as described above) may be transferred to the pad of a conventional 3.5 mini-patch. This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

When the compositions of the invention comprising hydroxychloroquine in combination with amodiaquine as the active ingredients are to be administered topically (epicutaneously), in the form of either a gel or a spray, the compositions preferably comprise hydroxychloroquine and amodiaquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form may be prepared from the starting ingredients set forth below:

| Hydroxychloroquine sulphate | 2400 | mg (12 200-mg tablets) |
|---|---|---|
| Amodiaquine hydrochloride | 800 | mg (4 200-mg tablets) |
| Lidocaine jelly 2% or ointment 5% | 10 ml or 10 mg, respectively | |
| Ethyl alcohol (70% aqueous solution) | 15 | ml |
| Peppermint spirit oil | 2.5 | ml |
| Water | 2.5 | ml |

Preferably, this composition is prepared by crushing the hydroxychloroquine sulphate tablets together with the amodiaquine hydrochloride tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a ‘variety of sources, including beauty supply shops); only warts that appear on the toes, fingers, elbows, knees and other hard surfaces of the patients skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

When the compositions of the invention comprising hydroxychloroquine in combination with amodiaquine as the active ingredients are to be administered transdermally, in the form of a patch, approximately 3.5 gm of the gel form (prepared as described above) may be transferred to the pad of a conventional 3.5 mini-patch. This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

When the compositions of the invention comprising chloroquine in combination with both hydroxychloroquine and amodiaquine as the active ingredients are to be administered topically (epicutaneously), in the form of either a gel or a spray, the compositions preferably comprise chloroquine, hydroxychloroquine and amodiaquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition is set forth below:

| | | |
|---|---|---|
| Chloroquine phosphate | 4000 | mg (8 500-mg tablets) |
| Hydroxychloroquine sulphate | 2400 | mg (12 200-mg tablets) |
| Amodiaquine hydrochloride | 800 | mg (4 200-mg tablets) |
| Lidocaine jelly 2% | 10 | ml |
| Ethyl alcohol (70% aqueous solution) | 15 | ml |
| Peppermint spirit oil | 2.5 | ml |
| Water | 2.5 | ml |

Preferably, this composition is prepared by crushing the chloroquine phosphate tablets together with both the hydroxychloroquine sulphate tablets and the amodiaquine hydrochloride tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops), only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

When the compositions of the invention comprising chloroquine in combination with both hydroxychloroquine and amodiaquine as the active ingredients are to be administered transdermally, in the form of a patch, approximately 3.5 gm of the gel form (prepared as described above) may be transferred to the pad of a conventional 3.5 mini-patch. This form of the medication is preferably administered to a patient in the same manner as the gel form, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart,

In-vivo experimental results which demonstrate the efficacy of the foregoing compositions are set forth below. Specifically, the following working examples illustrate both the manner in which a representative sample of the compositions of the present invention have been used in human subjects suffering from at least one wart associated with infections of the human papillomavirus, and the experimental results obtained, which demonstrate the efficacy of the invention.

### EXAMPLE 1

A juvenile male, specifically a 12-year old boy of Hispanic ancestry, was observed as having warts on three out of five fingers of the right hand. These warts were first filed in the manner set forth above for better contact with the medication, and they were then treated with a composition in gel form containing hydroxychloroquine as the only active ingredient (prepared in the manner set forth above for such compositions). This gel composition was applied to each of the warts, in an amount approximately equal to the surface area of each wart, once or twice a day for approximately one week, following which it was observed that all of the warts had disappeared completely, without leaving any visible scarring.

### EXAMPLE 2

Another juvenile male, specifically a 15-year old boy also of Hispanic ancestry, presented with warts on his fingers. The same composition as in Example 1 was applied, in the same manner and with the same frequency as in Example 1, and after approximately one week of such treatments, similar results were observed, that is. all of the warts had disappeared completely, without leaving any visible scarring.

### EXAMPLE 3

An adult male, specifically a 55-year old man of Hispanic ancestry, was observed to have genital warts, specifically, warts scattered about the skin of the scrotum. The same composition as in Example 1 was applied, in the same manner and with the same frequency as in Example 1, and after approximately one week of such treatments it was observed that 50% of the mass of the warts had disappeared. Thereafter, the patient switched to treatments with a composition in gel form containing chloroquine as the only active ingredient (prepared in the manner set forth above for such compositions), and after two days of similar twice-daily treatments with the latter cornposition, it was observed that the remaining 50% of the mass of the warts had disappeared.

### EXAMPLE 4

An adult female, specifically a 50-year old black woman, and her male partner, a 24-year old man of mixed Hispanic and black ancestry, both of whom presented with genital warts. The female, who was also living with a human immunodeficiency virus (HIV) infection, reported warts on her labia majora, which were then treated with a composition in gel form containing chloroquine as the only active ingredient (prepared in the manner set forth above for such compositions). This gel composition was applied to each of the warts, in an amount approximately equal to the surface area of each wart, once or twice a day for approximately four weeks, after which the patient reported that she observed a great deal of improvement. Thereafter, the female patient switched to treatments with a composition in gel form containing chloroquine and amodiaquine as the active ingredients (prepared in the manner set forth above for such compositions), and after approximately two weeks of similar twice-daily treatments with the latter composition, she reported that the warts had disappeared.

The same treatment regime was used on her male partner, who presented with warts on his penis, and who, in addition, reported having been born with HIV. After three months it was observed that the warts had disappeared completely. It is believed that this patient may have required a longer duration of treatment to achieve a successful result due to his underlying HIV infection, which possibly may have reduced the ability of his immune system to combat the human papillomavirus infection.

### EXAMPLE 5

Another adult male, specifically a 36-year old man of Hispanic ancestry, was observed as having warts on one of his fingers. The same composition as in Example 1 was applied, in the same manner and with the same frequency as in Example 1, and after two days of such treatments, similar results were observed; that is, all of the warts had disappeared completely, without any visible scarring.

### EXAMPLE 6

Another adult female, specifically a 45-year old woman of Hispanic ancestry, presented with warts on her fingers. These warts were treated with a composition in gel form containing chloroquine as the only active ingredient (prepared in the manner set forth above for such compositions), which was applied in the same manner as in Example 1. After three days of such treatments, the patient reported that all of the warts had disappeared completely, without any visible scarring.

### EXAMPLE 7

Another juvenile male, specifically a 16-year old boy also of Hispanic ancestry, presented with two warts on his hand. After filing, a composition in gel form containing amodiaquine as the only active ingredient (prepared in the manner set forth above for such compositions) was applied to each wart, in the same manner and with the same frequency as in Example 1, and after approximately one week of such treatments, the warts were reduced in size by approximately 50%. Following a second week of similar treatments, both of the warts had disappeared completely.

### EXAMPLE 8

Two juvenile females, specifically a 16-year old girl and her 14-year old sister, both also of Hispanic ancestry, each presented with warts on their feet -the 16-year old had two large warts on one foot, which made walking difficult for her without pain, while the 14-year old had one smaller wart on one of her feet. After filing, a composition in gel form containing the combination of chloroquine and hydroxychloroquine as the active ingredients (prepared in the manner set forth above for such compositions) was applied generously to all of the warts on the feet of both patients. After approximately three weeks of such treatments twice a day, all of the warts had disappeared completely from the feet of both patients.

### EXAMPLE 9

An adult male, specifically a 50-year old West African man from Senegal, presented with many different warts on his feet. After filing, a composition in gel form containing the combination of chloroquine, hydroxychloroquine and amodiaquine as the active ingredients (prepared in the manner set forth above for such compositions) was applied to the warts, as well as to the areas of his feet that were unaffected. After two weeks of such treatments twice a day, all of the warts had disappeared completely from both of his feet, allowing the patient to walk and wear shoes comfortably.

### EXAMPLE 10

An adult male, specifically a 50-year old West African man from Nigeria, was observed to have two (2) genital warts on the shaft of his penis. These warts were treated with a composition in gel form containing chloroquine as the only active ingredient (prepared in the manner set forth above for such compositions). This gel composition was applied to each of the warts, in an amount approximately equal to the surface area of each wart, once a day for two weeks, following which it was observed that both warts had disappeared completely, leaving the skin flat. This patient was observed as never having a recurrence of the warts.

### EXAMPLE 11

An adult male, specifically a 40-year old West African man from Nigeria, was observed to have a genital wart on the shaft of his penis. This wart was treated with a composition in gel form containing chloroqine and amodiaquine as the active ingredients (prepared in the manner set forth above for such compositions). This gel composition was applied to the wart, in an amount approximately equal to the surface area of the wart, and the area was then covered with an adhesive bandage, once a day for two weeks, following which it was observed that the wart had disappeared completely, leaving the skin flat. This patient was observed as never having a recurrence of the warts.

### EXAMPLE 12

A juvenile female, specifically a 15-year old girl of Nigerian ancestry, was observed as having two plantar warts (located on the bottom of her right foot). These warts were treated with a composition in gel form containing chlorogine and hydroxychloroquine as the active ingredients (prepared in the manner set forth above for such compositions). This gel composition was applied to each of the warts, in an amount approximately equal to the surface area of each wart, and the area was then covered with an adhesive bandage, once a day for three weeks, following which it was observed that both warts had disappeared completely, leaving the skin flat, This patient was observed as never having a recurrence of the warts.

Apart from the foregoing examples, in vitro experimental data have been obtained which strongly indicate that the compositions of the present invention are highly active in inhibiting replication of the human papillomavirus. Tests were conducted to assess the activity of each of chloroquine, hydroxychloroquine and amodiaquine, as compared with the activity of cidofovir, a known inhibitor of replication of the human papillornavirus (“HPV”).

Specifically, in these tests, vectors for expression of an HPV genotype-matched set of viral El and E2 proteins (derived from virus strain HPV-11) along with an HPV ori-containing plasmid were cotransfected into HEK293 cells. The cells were cultured in the absence or presence of the test compounds (chloroquine, hydroxychloroquine, and amodiaquine), each at 1, 10, and 100 μM concentrations. Low molecular weight DNA was harvested 2 days post-transfection and digested with Dpnl and exonuclease Ill to remove unreplicated transfected plasmid DNA. The replicated DNA was then subjected to real time qPCR (quantitative polymerase chain reaction) analyses in triplicate. Two controls were performed. One was to omit the El expression vector to provide a background amount of undigested and unreplicated DNA. The other, positive control was treatment with the known inhibitor cidofovir, as mentioned above. A toxicity assay based upon cell viability at the time of harvest on day 2 was performed alongside each transient replication assay in 293 cells. The test compounds were added 4 hours post transfection for a total exposure of 44 hours. More than 1×10⁵ cells were scored in a BioRad Automatic Cell Counter, with a determination of the total numbers, the numbers of trypan blue stained (dead) cells, and the % of dead cells.

The results of these tests are set forth below in Table 1, in which EC₅₀ represents the concentration of the tested compound that reduces viral replication by 50%, EC₉₀ represents the concentration of the tested compound that reduces viral replication by 90%, CC₅₀ represents the concentration of the tested compound that reduces cell viability by 50%, SI₅₀ represents CC/EC₅₀, and SI₉₀ represents CC₅₀/EC₉₀

TABLE 1

| Compound | EC₅₀ | EC₉₀ | CC₅₀ | SI₅₀ | SI₉₀ |
|---|---|---|---|---|---|
| Cidofovir | 148.00 | >200.00 | >200.00 | >1 | 1 |
| Chloroquine | 3.00 | 28.00 | >100.00 | >33 | >4 |
| Hydroxychloroquine | >100.00 | >100.00 | >100.00 | 1 | 1 |
| Amodiaquine | 5.00 | 58.00 | >100.00 | >20 | >2 |

Based on the experimental examples and test results summarized above, it is believed that the present invention comprises treatment methods and compositions that can treat infections caused by the human papillomavirus and can remove warts associated with that virus and prevent the recurrence of such warts, all by inhibiting replication of the virus. It is also believed that the removal of such warts, particularly the rectal and genital warts and those found in the oral cavity associated with the sexually transmitted types of the human papillomavirus, will reduce the risk of spreading that virus, and will therefore subsequently reduce the morbidity and mortality rates associated with cervical, vaginal, vulvar, penile, anal, rectal and/or oropharyngeal cancers. It is further believed that the methods and compositions of the present invention can be used as a prophylactic treatment, to provide protection during sexual intercourse from infections of the human papillomavirus for individuals who have not yet been infected.

It is also believed that the compositions and methods of the present invention may also be effective to treat infections in other mammalian species, caused by non-human papillomaviruses that may be specific to such species, and may be effective to remove warts associated with those viruses and to prevent the recurrence of such warts, all by inhibiting the replication of such non-human papillomaviruses in the same manner as with the human papillomavirus. This belief is based upon the close similarities among the various mammalian papillomaviruses, in terms of the organization of the viral genome, and in terms of the behavior of the viral proteins and the manner in which those proteins interact with host proteins. All papillomaviruses, regardless of type or the species they infect, need to modulate the host's immune system, which is accomplished with three viral proteins, designated ES, E6 and E7, which slightly modify the host's cells, making it difficult for the host's immune system to fight back.

Thus. since the target tissues, infection cycles and reproductive programs of all papillomaviruses are quite similar, independent of the papillomavirus type or the species infected, it is reasonable to conclude not only that all such viruses are dependent upon the same host properties and functions, but also that all such viruses would be susceptible to similar host inhibitors as well as pharmacological inhibitors, such as those which are disclosed herein. Accordingly, the applicant strongly believes that the compositions and methods of the present invention will be effective to treat infections of non-human papillomaviruses in other mammalian species, and to inhibit the replication of papillomaviruses in such species.

While there has been described what are at present considered to be the preferred embodiments of the present invention, it will be apparent to those skilled in the art that the embodiments described herein are by way of illustration and not of limitation. Various modifications of the disclosed embodiments, as well as alternative embodiments of the invention, will become apparent to persons skilled in the art upon reference to the description of the invention. Therefore, it is to be understood that various changes and modifications may be made in the embodiments disclosed herein without departing from the true spirit and scope of the present invention.

1. A method of treatment of an existing papillomavirus infection in a mammalian subject in need thereof, comprising administering to said mammalian subject a therapeutically effective amount of at least one compound selected from the group consisting of chloroquine, hydroxychloroquine, amodiaquine, and respective pharmaceutically acceptable salts thereof.

2. The method of treatment of claim 1 wherein said compound is optionally administered together with at least one pharmaceutically acceptable non-active ingredient.

3. The method of treatment of claims 1 or 2 wherein the papillomavirus infection is characterized by the presence of epithelial lesions,

4. A method of inhibiting or preventing the development of a papillomavirus infection in a mammalian subject comprising administering to said mammalian subject an antivirally effective amount of at least one compound selected from the group consisting of chloroquine, hydroxychloroquine, amodiaquine, and respective pharmaceutically acceptable salts thereof.

5. The method of treatment of claim 4 wherein said compound is optionally administered together with at least one pharmaceutically acceptable non-active ingredient.

6. A pharmaceutical composition for the treatment of a papillomavirus infection in a mammalian host, comprising a therapeutically effective amount of at least one compound selected from the group consisting of chloroquine, hydroxychloroquine, amodiaquine, and respective pharmaceutically acceptable salts thereof.

7. The pharmaceutical composition of claim 6 further optionally comprising at least one pharmaceutically acceptable non-active ingredient.

8. The pharmaceutical composition of claims 6 or 7 wherein the papillomavirus infection is characterized by the presence of epithelial lesions.

9. A method for the treatment of virally-induced tumors in mammals, wherein said tumors are associated with a papillomavirus, the method comprising the application of the composition of claim 6 or 7 to a mammal in need of such treatment.

10. A method of treatment of an existing papillomavirus infection in a human in need thereof, comprising administering to said human a therapeutically effective amount of at least one compound selected from the group consisting of chloroquine, hydroxychloroquine, amodiaquine, and respective pharmaceutically acceptable salts thereof.

11. The method of treatment of claim 10 wherein said compound is optionally administered together with at least one pharmaceutically acceptable non-active ingredient.

12. The method of treatment of claims 10 or 11 wherein the papillomavirus infection is characterized by the presence of epithelial lesions.

13. The method of treatment of claim 12 wherein the epithelial lesions are selected from one or more of the group consisting of verrucae warts, flat warts, plantar warts and anogenital warts of the skin and mucosal surfaces,

14. A method of inhibiting or preventing the development of a papillomavirus infection in a human comprising administering to said human an antivirally effective amount of at least one compound selected from the group consisting of chloroquine, hydroxychloroquine, amodiaquine, and respective pharmaceutically acceptable salts thereof.

15. The method of treatment of claim 14 wherein said compound is optionally administered together with at least one pharmaceutically acceptable non-active ingredient.

16. A pharmaceutical composition for the treatment of a papillomavirus infection in a human host, comprising a therapeutically effective amount of at least one compound selected from the group consisting of chloroquine, hydroxychloroquine, amodiaquine, and respective pharmaceutically acceptable salts thereof,

17. The pharmaceutical composition of claim 16 further optionally comprising at least one pharmaceutically acceptable non-active ingredient.

18. The pharmaceutical composition of claims 16 or 17 wherein the papillomavirus infection is characterized by the presence of epithelial lesions.

19. The pharmaceutical composition of claim 18 wherein the epithelial lesions are selected from one or more of the group consisting of verrucae warts, flat warts, plantar warts and anogenital warts of the skin and mucosal surfaces.

20. A method of treating a viral disease selected from the group consisting of anogenital warts, verrucae warts, flat warts, plantar warts and papillomavirus in a human being, the method comprising the administration of the composition of claims 16 or 17 to a human being in need of such treatment.

21. A method for the treatment of virally-induced tumors in humans, wherein said tumors are associated with the human papillomavirus, the method comprising the application of the composition of claims 16 or 17 to a human in need of such treatment.

22. A method for the treatment of virally-induced tumors in humans, wherein said tumors are associated with the human papillomavirus and are selected from the group consisting of verrucae warts, flat warts, plantar warts and anogenital warts, the method comprising the application of the composition of claims 16 or 17 to a human in need of such treatment.

23. A method of inhibiting the replication of a papillomavirus in a cell infected with said virus, the method comprising exposing the cell infected with said virus to an antivirally effective amount of at least one compound selected from the group consisting of chloroquine, hydroxychloroquine, amodiaquine, and respective pharmaceutically acceptable salts thereof.

24. The method of claim 23 wherein said exposing step comprises culturing said cell in the presence of said at least one compound.

25. A method of treating a papillomavirus infection in a cell, the method comprising exposing the cell infected with said virus to an antivirally effective amount of at least one compound selected from the group consisting of chloroquine, hydroxychloroquine, amodiaquine, and respective pharmaceutically acceptable salts thereof.

26. The method of claim 25 wherein said exposing step comprises culturing said cell in the presence of said at least one compound.
